(19) United States
(12) Patent Application Publication
(10) Pub. No.: US 2010/0178690 A1
(43) Pub. Date: Jul. 15, 2010

(54) BIOMOLECULE DETECTION APPARATUS AND BIOMOLECULE MEASUREMENT SYSTEM

(75) Inventors: Changsung Sean KIM, Yongin (KR); Ji Hye Shim, Suwon (KR)

Correspondence Address: MCDERMOTT WILL & EMERY LLP, 18191 VON KARMAN AVE., SUITE 500, IRVINE, CA, 92612-7108, US

(73) Assignee: SAMSUNG ELECTRO-MECHANICS CO., LTD., Suwon (KR)

(21) Appl. No.: 12/501,352

(22) Filed: Jul. 10, 2009

(30) Foreign Application Priority Data
Jan. 13, 2009 (KR) 10-2009-0002786
Jan. 13, 2009 (KR) 10-2009-0002787

(51) Int. Cl. C12M 1/34

(52) U.S. Cl. 435/287.2; 435/287.1; 435/288.7

(57) ABSTRACT

The present invention relates to a biomolecule detection apparatus and a biomolecule measurement system. A biomolecule detection apparatus according to an aspect of the invention may include: an upper disc having a fluid inlet in a thickness direction through which a fluid is introduced to the inside; a lower disc laminated to the upper disc and having a fluid outlet in a thickness direction through which the fluid exits to the outside; detection units provided on each of the upper disc and the lower disc and including spherical microbeads having surfaces coated with materials used to capture biomolecules; and via holes provided along the edge of each of the upper disc and the lower disc so that the fluid flows between the upper disc and the lower disc.

According to the present invention, a biomolecule detection apparatus that achieves excellent performance in the detection of rare cells and has a biochip structure to ensure quality mass production, and a biomolecule measurement system that can accurately and easily measure the biomolecules captured by the biomolecule detection apparatus can be provided.

### CROSS-REFERENCE TO RELATED APPLICATIONS

This application claims the priority of Korean Patent Application Nos. 2009-0002786 and 2009-0002787 filed on Jan. 13, 2009, in the Korean Intellectual Property Office, the disclosure of which is incorporated herein by reference.

### BACKGROUND OF THE INVENTION

1. Field of the Invention

The present invention relates to a biomolecule detection apparatus and a biomolecule measurement system, and more particularly, to a biomolecule detection apparatus that achieves excellent performance in the detection of rare cells and has a biochip structure to ensure quality mass production and a biomolecule measurement system.

2. Description of the Related Art

There has been an increasing demand for methods and apparatuses for detecting target molecules, such as DNA and RNA, specific proteins or rare cells in blood recently in the biotechnology industry. For example, there may be only one circulating tumor cell (CTC) for every 1 billion blood cells. Techniques for detecting the presence of CTCs are required for the early diagnosis of diseases such as cancer. However, since rare cells, such as CTCs, exist at extremely low concentrations in blood, it is currently extremely difficult to accurately and quickly detect rare cells and count the number of detected rare cells. Thus, in the art, research has been conducted on various kinds of methods of detecting rare cells.

### SUMMARY OF THE INVENTION

An aspect of the present invention provides a biomolecule detection apparatus that achieves excellent performance in the detection of rare cells and has a biochip structure to ensure quality mass production.

Another aspect of the present invention provides a biomolecule measurement system that can accurately measure the amount of biomolecules detected by the biomolecule detection apparatus.

According to an aspect of the present invention, there is provided a biomolecule detection apparatus including: an upper disc having a fluid inlet in a thickness direction through which a fluid is introduced to the inside; a lower disc laminated to the upper disc and having a fluid outlet in a thickness direction through which the fluid exits to the outside; detection units provided on each of the upper disc and the lower disc and including spherical microbeads having surfaces coated with materials used to capture biomolecules; and via holes provided along the edge of each of the upper disc and the lower disc so that the fluid flows between the upper disc and the lower disc.

The microbeads may include a plurality of microbeads having different diameters.

Each of the detection units may be divided into a plurality of sections each having microbeads of the same diameter among the plurality of microbeads.

The microbeads, provided in the plurality of sections of the upper disc, may increase in diameter toward the center of the upper disc.

The microbeads, provided in the plurality of sections of the lower disc, may decrease in diameter toward the center of the lower disc.

The detection units may include a plurality of detection units and be separated from each other.

The plurality of detection units may be separated from each other in a clockwise direction.

The materials used to capture the biomolecules may include one material selected from the group consisting of antigens, antibodies and conductive polymers.

The microbeads may include a plurality of microbeads arranged in columns and rows and be arranged in a matrix array.

The microbeads may include a plurality of microbeads while the microbeads disposed adjacent to each other are in contact with each other.

The microbeads may be optically exposed to the outside of the upper and lower discs.

The microbeads may include a light-transmissive material.

Each of the microbeads may have a diameter of 5 μm or greater.

Each of the microbeads may have a diameter ranging from 5 μm to 200 μm.

The fluid inlet and the fluid outlet, provided in the thickness directions of the upper and lower discs, respectively, may face each other.

Each of the upper and lower discs may include a transparent material.

According to another aspect of the present invention, there is provided a biomolecule measurement system including: an upper disc having a fluid inlet in a thickness direction through which a fluid is introduced to the inside, a lower disc laminated to the upper disc and having a fluid outlet in a thickness direction through which the fluid exits to the outside, detection units provided on each of the upper disc and the lower disc and including spherical microbeads having surfaces coated with materials used to capture biomolecules, and via holes provided along the edge of each of the upper disc and the lower disc so that the fluid flows between the upper disc and the lower disc; and a measurement apparatus having an optical device measuring the biomolecules captured by the biomolecule detection apparatus.

The biomolecules may be coupled to fluorescent materials.

The optical device may include a CCD camera and a lens disposed above or under a detection area.

An image, obtained by capturing the detection area with the CCD camera, may be divided into portions arranged in a lattice pattern and stored in a processing unit.

The biomolecule measurement system may further include a stage receiving the biomolecule detection apparatus and moving in one direction and another direction at right angles to the first direction.

The optical device may be an optical pickup device including a laser diode and a light receiving unit and arranged above or under the detection area.

The optical pickup device can move in one direction, and the biomolecule measurement system further includes a stage receiving the biomolecule detection apparatus and enabling the rotary motion of the biomolecule detection apparatus.

According to another aspect of the present invention, there is provided a biomolecule detection apparatus including: a body unit having a detection area therein; a fluid inlet and a fluid outlet provided on one surface and the other surface of the body, respectively, so that a fluid can pass through the detection area; and spherical microbeads disposed in the detection area and having surfaces coated with materials used to capture biomolecules.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view illustrating a biomolecule detection apparatus according to an exemplary embodiment of the invention;

FIG. 2 is an upper plan view illustrating how biomolecules are detected using the microbeads of FIG. 1;

FIG. 3 is a perspective view illustrating how biomolecules are detected using the microbeads of FIG. 1;

FIG. 4 is a perspective view illustrating a biomolecule detection apparatus according to another exemplary embodiment of the invention;

FIG. 5 is a perspective view illustrating a biomolecule detection apparatus according to an exemplary embodiment of the invention;

FIG. 6 is an exploded perspective view illustrating the biomolecule detection apparatus of FIG. 5;

FIG. 7A is a plan view illustrating an upper disc of the biomolecule detection apparatus of FIG. 5;

FIG. 7B is a plan view illustrating a lower disc of the biomolecule detection apparatus of FIG. 5;

FIG. 8 is a detailed plan view illustrating a detection unit that is employed in an embodiment of the invention;

FIG. 9 is a plan view illustrating a detection unit of a biomolecule detection apparatus according to another exemplary embodiment of the invention;

FIG. 10 is a view illustrating a biomolecule measurement system according to another exemplary embodiment of the invention;

FIG. 11 is a view illustrating a biomolecule measurement system according to another exemplary embodiment of the invention; and

FIG. 12 is a cross-sectional view illustrating an optical pickup device of FIG. 10 in detail.

### DETAILED DESCRIPTION OF THE PREFERRED EMBODIMENT

Exemplary embodiments of the present invention will now be described in detail with reference to the accompanying drawings.

The invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

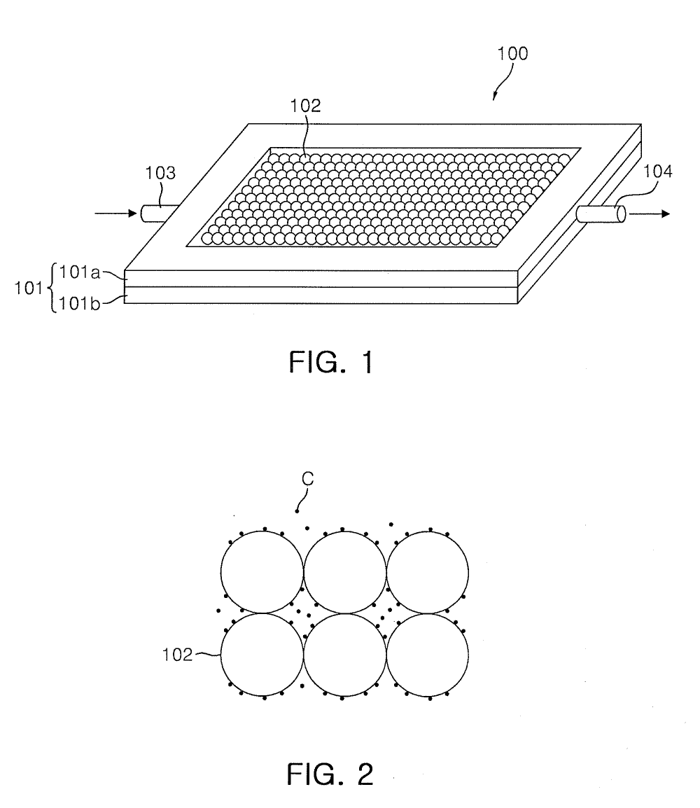

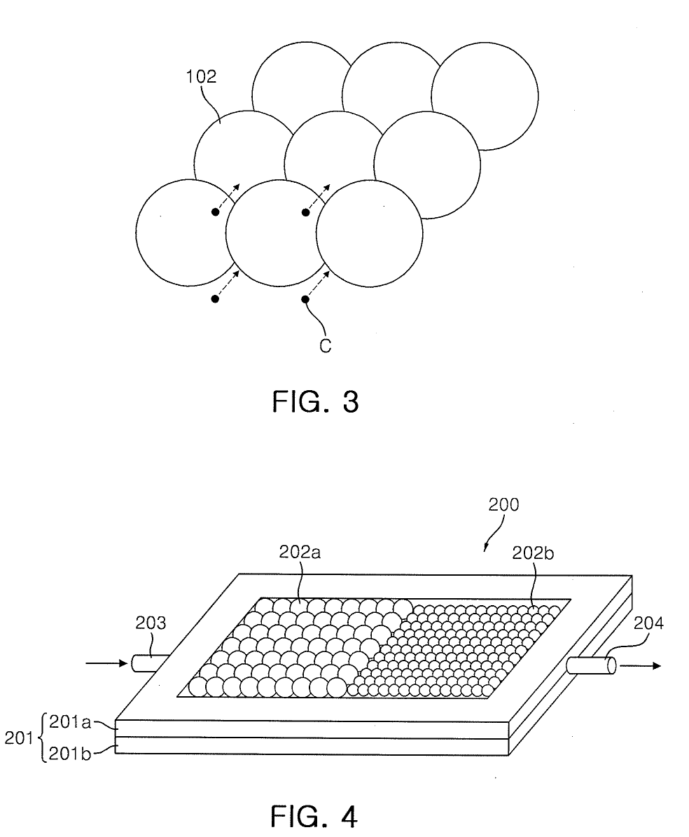

FIG. 1 is a perspective view illustrating a biomolecule detection apparatus according to an exemplary embodiment of the invention. FIG. 2 is an upper plan view illustrating how biomolecules are detected using the microbeads of FIG. 1. FIG. 3 is a perspective view illustrating how biomolecules are detected using the microbeads of FIG. 1. First, referring to FIG. 1, a biomolecule detection apparatus 100 according to this embodiment includes a body unit 101, microbeads 102, a fluid inlet 103 and a fluid outlet 104. Specific materials among biomolecules included in a fluid, such as blood, which is injected through the fluid inlet 103, are captured by the microbeads 102 that form a detection area, and the remaining fluid exits through the fluid outlet 104.

The body unit 101 includes a detection area for detecting biomolecules while blood passes therethrough. As shown in FIG. 1, the body unit 101 may consist of an upper body 101a and a lower body 101b. The detection area may be configured in such a manner that a plurality of microbeads 102 are disposed within grooves formed in the upper body 101a and the lower body 101b. Alternatively, grooves may be formed in any one of the upper body 101a and the lower body 101b, which is then covered with a transparent cover. Here, as the microbeads 102, serving as a biomolecule capturing unit, are optically exposed to the outside, biomolecules can be measured using an optical device. Though not shown explicitly in FIG. 1, the microbeads 102 may have a configuration so that biomolecules can be measured from below the body unit 101. That is, a hole may be drilled through the center of the lower body 101b, which is then covered with a lower transparent cover. Alternatively, the lower body 101b itself may be formed of a transparent material. Therefore, measurement can be performed both above and below the body unit 101 to thereby increase measurement speed. Further, this invention is not limited thereto. As shown in FIG. 1, the body unit 101 is a flat panel in the form of a biochip that allows for easy handling for measurement.

The fluid inlet 103 and the fluid outlet 104 of the biomolecule detection apparatus 100 may be flow passages serving as an inlet and an outlet through which a fluid, such as blood, enters and exits. To this end, the fluid inlet 103 and the fluid outlet 104 may be formed on two surfaces of the body unit 101 that face each other. However, the locations of the fluid inlet 103 and the fluid outlet 104 may vary according to embodiments of the invention.

The microbeads 102 are spherical and serve as obstacles to block the flow of a fluid, such as blood. In this embodiment, the detection area may be formed in such a way that the microbeads 102 are separately manufactured and injected into the body unit 101. Here, the obstacles are not made by partially removing the inside of the body unit 101 by etching. As such, since an etching process is not carried out, biochips can be manufactured using mass-production techniques.

As shown in FIGS. 2 and 3, when blood passes through the detection area, predetermined biomolecules C, such as target molecules (DNA or RNA), specific proteins, rare cells like circulating tumor cells (CTCs), can be captured onto the surface of the microbeads 102. To this end, the surface of the microbeads 102 is coated with materials allowing for antigen-antibody interactions. Specifically, the surface of the microbeads 102 may be coated with appropriate materials according to the kind of biomolecules to be detected, for example, an antigen or an antibody. The surface of the microbeads 102 may be coated with conductive polymers.

When the biomolecules to be detected are CTCs, the surface of the microbeads 102, serving as a capturing unit, may be coated with chemical substances or biomolecules that specifically react to CTCs. For example, an anti-EpCAM (epithelial cell adhesion molecule) that has an antigen-antibody interaction specifically with an EpCAM on the surface of the CTC or a DNA or RNA aptamer having a bond strength equal to or greater than that of an anti-EpCAM. In addition to the EpCAM, biological or chemical substances that are expressed on the surface of the CTCs and are specifically bonded to specific proteins differentiated from other nucleated cells, such as leukocytes, can be used. In order to efficiently coat the CTC surface with biomolecules or chemical substances, functional groups, such as an amine group (—NH2), a carboxyl group (—COOH) and a thiol group (—SH), may be located at regular intervals on the surface of the microbeads 102. These functional groups form strong covalent bonds, including amid bonds or disulfide bonds, with biomolecules, such as antibodies, and aptamers, such as DNA, so that the capturing unit is firmly secured onto the surface of the microbeads 102. Here, the intervals between the functional groups may be adjusted to thereby increase detection efficiency.

As shown in FIG. 1, the microbeads 102 are presented in a matrix format comprising columns and rows within the planar body unit 101. Further, the microbeads 102, disposed adjacent to each other, may be in contact with each other. In this configuration, a large number of microbeads 102 can be provided in the detection area to thereby increase detection efficiency. Here, even when the microbeads 102 are in contact with each other, as shown in FIG. 3, an area large enough for the biomolecules C to pass therethrough is ensured both above and below the microbeads 102 in contact with each other.

Since spheres have a greater contact area with a fluid than other solids, such as cylinders and prisms, the biomolecules C can be more efficiently detected using the microbeads 102. Here, in order to observe all the biomolecules C attached to the spherical surfaces with the naked eye or under a microscope while the microbeads 102 are located within the body unit 101, the microbeads 102 are preferably formed of transparent materials. Examples of the transparent materials may include polymers, silica and glass. These transparent materials are selected in consideration of antibody coating as well as transmittance.

Furthermore, since the microbeads 102 are spherical, a reduction in shear stress increases the possibility of capturing biomolecules. That is, spherical microbeads, contacting a flowing fluid, have lower shear stress in an area contacting the fluid than cylindrical or prismatic microbeads. This reduction in shear stress can reduce the possibility that cells, such as CTCs, having been captured by the microbeads 102, may be swept away by the flow of the fluid to rejoin the fluid stream. Therefore, the possibility of detecting desired biomolecules C can be increased.

The size of the microbeads 102 can vary according to the size of target biomolecules. However, when the microbeads 102 are small, cell capturing performance can be improved since a larger number of microbeads 102 can be provided in the detection area of the same size. However, when the diameter of the microbead 102 is excessively small, the area through which blood passes becomes smaller. Thus, rare cells, such as CTCs, which are relatively large, may not pass. Therefore, preferably, the diameter of the microbeads 102 may be approximately 5 μm or greater, and more preferably, within the range from 5 μm to 200 μm.

FIG. 4 is a perspective view illustrating a biomolecule detection apparatus according to another exemplary embodiment of the invention. A biomolecule detection apparatus 200 according to this embodiment is the same as the embodiment, shown in FIG. 1, in that the biomolecule detection apparatus 200 according to this embodiment includes a body unit 201, microbeads 202a and 202b, a fluid inlet 203 and a fluid outlet 204, and the body unit 201 may consist of an upper body 201a and a lower body 201b. This embodiment is different from the above-described embodiment in that the microbeads 202a and 202b vary in diameter. That is, as shown in FIG. 4, a detection area is divided into two different zones including the microbeads 202a and the microbeads 202b having a smaller diameter than the microbeads 202a. Here, the microbeads 202a may increase in size toward the fluid inlet 203. Alternatively, the microbeads 202a may decrease in size toward the fluid inlet 203. Though not shown, the detection area may be divided into three or more zones consisting of microbeads having different diameters. Like this embodiment, biomolecules of varying size can be filtered in stages by varying the diameter of each of the microbeads 202a and 202b and arranging the microbeads 202a and 202b in stages according to the fluid flow.

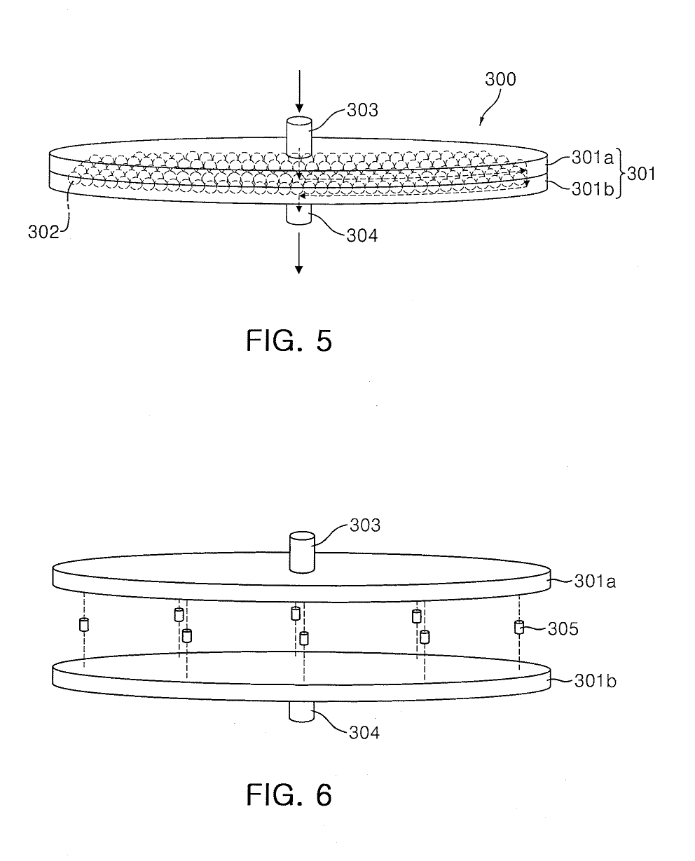

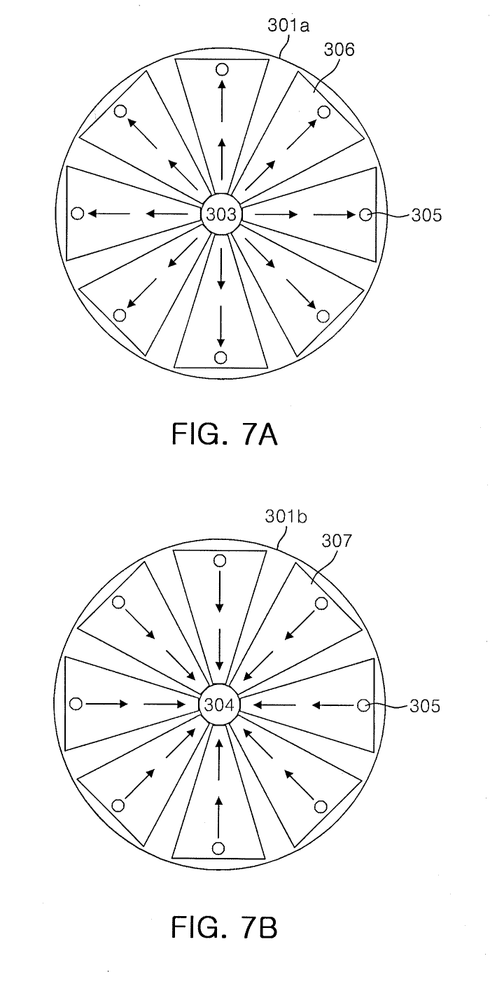

FIG. 5 is a perspective view illustrating a biomolecule detection apparatus according to another exemplary embodiment of the invention. FIG. 6 is an exploded perspective view illustrating the biomolecule detection apparatus of FIG. 5. FIGS. 7A and 7B are plan views illustrating an upper disc and a lower disc of the biomolecule detection apparatus of FIG. 5, respectively. A biomolecule detection apparatus 300 according to this embodiment includes a body unit 301, microbeads 302, a fluid inlet 303 and a fluid outlet 304. Specific substances among biomolecules included in a fluid, such as blood, injected through the fluid inlet 303, are captured by the microbeads 302 forming a detection area, and the remaining fluid exits through the fluid outlet 304. The body unit 301 includes an upper disc 301a and a lower disc 301b that have a compact disc-shaped structure and are laminated to one another. The upper and lower discs 301a and 301b include detection units 306 and 307, respectively. As the body unit 301 has a disc structure, the biomolecule detection apparatus 300 can be used in the form of a biochip that allows for easy handling for measurement.

Referring to FIGS. 7A and 7B, blood, injected through the fluid inlet 303, passes through the detection units 306 of the upper disc 301a and the detection units 307 of the lower disc 301b, and then exits through the fluid outlet 304. Here, biomolecules, captured onto the detection units 306 and 307, can be measured, which will be described below. In FIGS. 7A and 7B, the flow of blood is shown by arrow. In order to measure the captured biomolecules, the upper disc 301a and the lower disc 301b may be formed of transparent materials so as to be optically exposed to the outside. As such, the blood flow has a three-dimensional flow structure, that is, blood sequentially passes through the upper disc 301a and the lower disc 301b. As a result, the detection area for detecting target biomolecules may increase. To this end, via holes 305 are provided along the edges of the upper disc 301a and the lower disc 301b such that the fluid flows between the upper disc 301a and the lower disc 301b.

The fluid inlet 303 and the fluid outlet 304 of the biomolecule detection apparatus 300 may have flow passages serving as an inlet and an outlet through which a fluid, such as blood, enters and exits. Further, in order to induce a three-dimensional flow of blood, as described above, the fluid inlet 303 and the fluid outlet 304 may face each other in the thickness directions of the upper disc 301a and the lower disc 301b, respectively. However, the locations of the fluid inlet 303 and the fluid outlet 304 may be appropriately changed according to various embodiments of the invention.

The detection units 306 and 307 are provided on the upper disc 301a and the lower disc 301b, respectively, to detect specific biomolecules while blood is flowing. Here, in consideration of detection efficiency and measurement convenience, each of the detection units 306 and 307 may be divided into a plurality of sections. That is, as shown in FIGS. 7A and 7B, the plurality of detection units 306 and the plurality of detection units 307 are provided on the upper disc 301a and the lower disc 301b, respectively. Further, each of the plurality of the detection units 306 and 307 may be separated from each other in a clockwise direction.

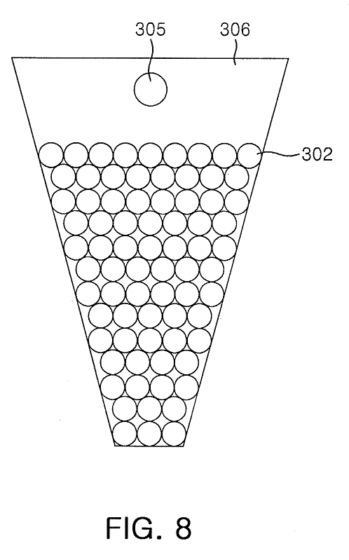

The detection units 306 and 307 include microbeads in order to capture biomolecules. FIG. 8 is a detailed plan view illustrating a detection unit that is employed in the embodiment of the invention. FIG. 8 illustrates the detection unit 306 of the upper disc 301a. The fluid, injected through the fluid inlet 303, passes through the microbeads 302 and reach the lower disc 301b through the via holes 305. Hereinafter, a description will be made on the basis of the upper disc, but this description can also be made on the basis of the lower disc. Further, the microbeads 302 may be the same as the microbeads 102, used in the embodiment of FIG. 1. Thus, the descriptions, made with reference to FIGS. 2 and 3, can be directly applied thereto.

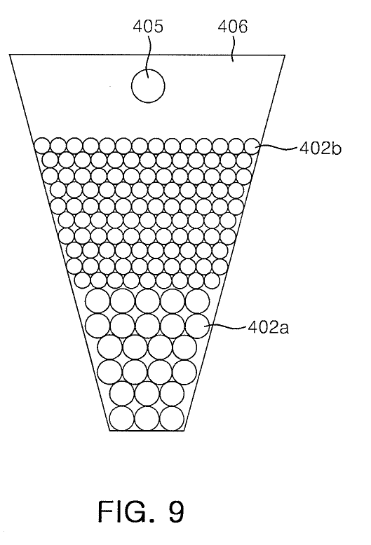

FIG. 9 is a plan view illustrating a detection unit of a biomolecule detection apparatus according to another exemplary embodiment of the invention. This embodiment is different from the previous embodiment in that microbeads 402a and 402b may have different diameters within a single detection unit 406. That is, as shown in FIG. 9, a detection area may be divided into two zones including the microbeads 402a and 402b while the microbeads 402a are larger in diameter than the microbeads 402b. Here, the microbeads 402a may increase in size toward the fluid inlet 403, that is, the central region. Alternatively, the microbeads 402a may decrease in size toward the central region. Though not shown, microbeads may be arranged in a reverse manner on detection units included in the lower disc. Alternatively, the detection area may be divided into three or more zones including microbeads having different diameters. Like this embodiment, biomolecules of varying size can be filtered in stages by varying the diameters of the microbeads 402a and 402b and arranging the microbeads 402a and 402b by stages according to the fluid flow.

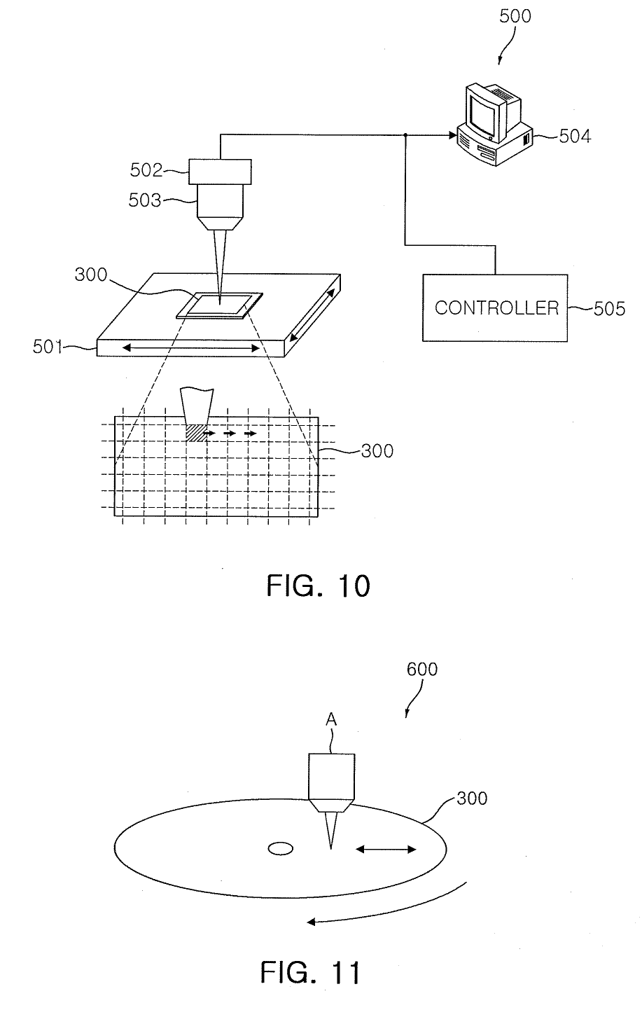

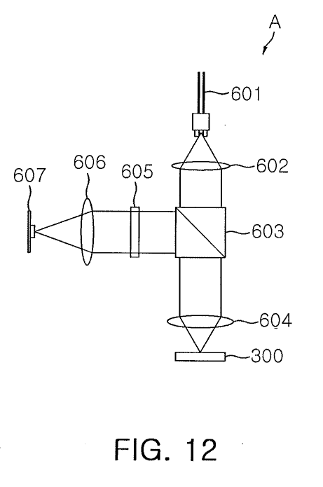

The biomolecule detection apparatus having the above-described configuration can easily perform measurement with the naked eye or under a microscope. However, measurement can be performed more accurately using an optical device. FIGS. 10 and 11 are views illustrating a biomolecule measurement system according to another embodiment of the invention. FIG. 12 is a cross-sectional view illustrating an optical pickup device of FIG. 10. First, as shown in FIG. 10, a biomolecule measurement system 500 includes a biomolecule detection apparatus 300 containing a sample, a stage 501, a CCD camera 502, an object lens 503, a processing unit 504 and a controller 505.

The biomolecule detection apparatus 300 has a configuration according to the embodiment of FIG. 5, that is, a biochip structure. Here, the biomolecule detection apparatus 300 contains a sample in which predetermined biomolecules are captured by passing blood therethrough. Here, in order to improve detection efficiency, phosphors and biomolecules may be coupled to each other. As for this technique for coupling phosphors to biomolecules, a known method in the art may be used.

As shown in FIG. 10, the stage 501 receives the biomolecule detection apparatus 300 on an upper surface thereof. The stage 501 can move from left to right and top to bottom. With this movement, an area to be detected is divided into portions arranged in a lattice pattern, and divided portions are saved as different individual images. Then, the number of fluorescent images is only counted among the stored images. This procedure is performed by the processing unit 504 connected to the CCD camera 502. The movement of the stage 501 can be controlled using the controller 505 that is connected to the processing unit 504. Here, the CCD camera 502 may be moved instead of the stage 501 according to embodiments.

Referring to FIGS. 11 and 12, a biomolecule measurement system 600 according to another exemplary embodiment of the invention uses an optical pickup device A. That is, the biomolecule measurement system 600 includes a biomolecule detection apparatus 300, a light source 601, such as a laser diode, a collimating lens 602, a beam splitter 603, an object lens 604, a dichroic filter 605, a sensor lens 606 and a light receiving lens 607. Here, a stage (not shown) that receives and rotates the disc-type biomolecule detection apparatus 300 may further be provided. While the optical pickup device A performs a linear movement in one direction, the optical pickup device A counts the number of fluorescent substances over the entire area of the biomolecule detection apparatus 300 so as to measure the amount of desired biomolecules.

Here, the system that measures biomolecules, detected by the biomolecule detection apparatus having the disc structure according to the embodiment to FIG. 5, is described with reference to FIGS. 10 through 12. However, biomolecules, detected by the biomolecule detection apparatus according to the embodiment of FIG. 1, can also be measured by employing a similar system.

As set forth above, according to exemplary embodiments of the invention, a biomolecule detection apparatus can achieve excellent performance in the detection of rare cells and has a biochip structure to ensure quality mass production, and a measurement system can accurately measure biomolecules captured by the biomolecule detection apparatus. When the biomolecule detection apparatus according to the embodiment is used, detection performance can be improved by inducing a three-dimensional fluid flow.

While the present invention has been shown and described in connection with the exemplary embodiments, it will be apparent to those skilled in the art that modifications and variations can be made without departing from the spirit and scope of the invention as defined by the appended claims.

What is claimed is:

1. A biomolecule detection apparatus comprising:
an upper disc having a fluid inlet in a thickness direction through which a fluid is introduced to the inside;
a lower disc laminated to the upper disc and having a fluid outlet in a thickness direction through which the fluid exits to the outside;
detection units provided on each of the upper disc and the lower disc and including spherical microbeads having surfaces coated with materials used to capture biomolecules; and
via holes provided along the edge of each of the upper disc and the lower disc so that the fluid flows between the upper disc and the lower disc.

2. The biomolecule detection apparatus of claim 1, wherein the microbeads comprise a plurality of microbeads having different diameters.

3. The biomolecule detection apparatus of claim 2, wherein each of the detection units is divided into a plurality of sections each having microbeads of the same diameter among the plurality of microbeads.

4. The biomolecule detection apparatus of claim 3, wherein the microbeads, provided in the plurality of sections of the upper disc, increase in diameter toward the center of the upper disc.

5. The biomolecule detection apparatus of claim 3, wherein the microbeads, provided in the plurality of sections of the lower disc, decrease in diameter toward the center of the lower disc.

6. The biomolecule detection apparatus of claim 1, wherein the detection units comprise a plurality of detection units and are separated from each other.

7. The biomolecule detection apparatus of claim 6, wherein the plurality of detection units are separated from each other in a clockwise direction.

8. The biomolecule detection apparatus of claim 1, wherein the materials used to capture the biomolecules comprise one material selected from the group consisting of antigens, antibodies and conductive polymers.

9. The biomolecule detection apparatus of claim 1, wherein the microbeads comprise a plurality of microbeads arranged in columns and rows and are arranged in a matrix array.

10. The biomolecule detection apparatus of claim 1, wherein the microbeads comprise a plurality of microbeads while the microbeads disposed adjacent to each other are in contact with each other.

11. The biomolecule detection apparatus of claim 1, wherein the microbeads are optically exposed to the outside of the upper and lower discs.

12. The biomolecule detection apparatus of claim 1, wherein the microbeads comprise a light-transmissive material.

13. The biomolecule detection apparatus of claim 1, wherein each of the microbeads has a diameter of 5 μm or greater.

14. The biomolecule detection apparatus of claim 1, wherein each of the microbeads has a diameter ranging from 5 μm to 200 μm.

15. The biomolecule detection apparatus of claim 1, wherein the fluid inlet and the fluid outlet, provided in the thickness directions of the upper and lower discs, respectively, face each other.

16. The biomolecule detection apparatus of claim 1, wherein each of the upper and lower discs comprises a transparent material.

17. A biomolecule measurement system comprising:
a biomolecule detection apparatus including an upper disc having a fluid inlet in a thickness direction through which a fluid is introduced to the inside, a lower disc laminated to the upper disc and having a fluid outlet in a thickness direction through which the fluid exits to the outside, detection units provided on each of the upper disc and the lower disc and including spherical microbeads having surfaces coated with materials used to capture biomolecules, and via holes provided along the edge of each of the upper disc and the lower disc so that the fluid flows between the upper disc and the lower disc; and
a measurement apparatus having an optical device measuring the biomolecules captured by the biomolecule detection apparatus.

18. The biomolecule measurement system of claim 17, wherein the biomolecules are coupled to fluorescent materials.

19. The biomolecule measurement system of claim 17, wherein the optical device comprises a CCD camera and a lens disposed above or under a detection area.

20. The biomolecule measurement system of claim 19, wherein an image, obtained by capturing the detection area with the CCD camera, is divided into portions arranged in a lattice pattern and stored in a processing unit.

21. The biomolecule measurement system of claim 19, further comprising a stage receiving the biomolecule detection apparatus and moving in one direction and another direction at right angles to the first direction.

22. The biomolecule measurement system of claim 17, wherein the optical device is an optical pickup device including a laser diode and a light receiving unit and arranged above or under the detection area.

23. The biomolecule measurement system of claim 22, wherein the optical pickup device can move in one direction, and
the biomolecule measurement system further comprises a stage receiving the biomolecule detection apparatus and enabling the rotary motion of the biomolecule detection apparatus.

24. A biomolecule detection apparatus comprising:
a body unit having a detection area therein;
a fluid inlet and a fluid outlet provided on one surface and the other surface of the body, respectively, so that a fluid can pass through the detection area; and
spherical microbeads disposed in the detection area and having surfaces coated with materials used to capture biomolecules.
